(19) United States
(12) Patent Application Publication
(10) Pub. No.: US 2026/0360564 A1
(43) Pub. Date: Nov. 12, 2026

(54) SYSTEM AND METHOD TO FACILITATE CONSULTATION IN RADIOLOGY

(71) Applicant: KONINKLIJKE PHILIPS N.V., EINDHOVEN (NL)

(72) Inventors: Qianxi LI, CAMBRIDGE, MA (US); Merlijn SEVENSTER, HAARLEM (NL); Hildebrandus Johannes LAMB, LEIDEN (NL); Sandra VOSBERGEN, LIEMPDE (NL); Peter Jacobus HAIMA, EINDHOVEN (NL); Mark Anthony HENNESSAY, NOORD BRABANT (NL)

(21) Appl. No.: 19/138,010

(22) PCT Filed: Dec. 4, 2023
(86) PCT No.: PCT/EP2023/084031

§ 371 Date: Jun. 11, 2025

Related U.S. Application Data
(60) Provisional application No. 63/432,018, filed on Dec. 12, 2022.

(51) Int. Cl. G16H 80/00; G16H 10/60

(52) U.S. Cl. CPC G16H 80/00; G16H 10/60

(57) ABSTRACT

Systems, apparatuses, and methods provide for matching patient data to individual patients. For example, such operations include determining data requirements based on content of a consultation request for a consultation. Missing information is identified based on the determined data requirements by retrieving available data from a data source and identifying unavailable data based on the retrieved available data and the determined data requirements. Summaries are generated by extracting clinical information relevant to performing the consultation from the retrieved available data. The summaries are prioritized and the identified unavailable data is highlighted in context of the consultation request.

### FIELD

The following relates generally to medical records. More particularly, embodiments herein relate to managing patient data for radiologist consultation support.

### BACKGROUND

In patient care environments there is often patient data coming from various sources. Often, healthcare platforms (e.g., Performance Management Data Platform, Virtualized Imaging Solution, Interoperability Solution, Electronic Medical Record (EMR), radiology information systems (RIS), picture archiving and communication system (PACS), etc.) typically have information about patients.

For example, in a radiology context, radiology information systems (RIS) have information about a patient, picture archiving and communication system (PACS) has all of the images, machine logs have the information about the imaging protocols and timestamps of various activities that happen while that patient was on the machine. But none of these systems create a complete record of the patient care.

In general, data for the same patient is often kept in different information silos and these separate bucket of data are not typically being coordinated for use by a radiologist. Current methods of pulling the patient data from various sources is often done manually by sifting through logs, phone calls, conversations with different people in the hospital. Such operations are laborious and prone to human error.

In addition to the primary task of integrating clinical information, image data and clinical interpretation, radiologists also shoulder the responsibilities to provide consultation for technologists, referral physicians, radiologist residents, physicians from other disciplines, and more. Research indicates that these activities constitute more than 26% of the radiologists' time while the image interpretation tasks constitute about 37%.

The following discloses certain improvements to overcome these problems and others.

### SUMMARY

As discussed above, it is currently challenging to provide complete data to radiologists for consultation tasks. The consultation tasks primarily include solving problems related to clinical management of patients, study reviews, answering order questions, image quality check, protocol request, procedure questions, protocoling, and/or the like, for which radiologists would refer to the clinical context of the study and patient. For all these examples, clinical context of the study and patient is typically utilized to perform these tasks.

While clinical decision support systems have been developed to improve the efficiency and quality of report interpretation, there is a lack of a system to facilitate the consultation tasks. Radiologists are often presented with incomplete or missing information or excess of irrelevant or fragmented data, which requires time to navigate to the information of interest or additional communication.

Similar to the report interpretation, the ability to appropriately collect, distill and interpret patient information is often useful for providing quality consultation. Radiologists, however, are often presented with incomplete or missing information or an excess of partially irrelevant data from a variety of sources which often requires time to collect and synthesize the clinical data.

For example, the need for clinical information in order to provide appropriate consultation varies depending on the specific consultation task. For protocolling, radiologists often utilize patient demographics information (e.g., height, weight), lab data (e.g., estimated glomerular filtration rate (eGFR)), patient's current and prior clinical context in order to get a holistic view of the patient. For image quality checks, radiologist would typically access to the Digital Imaging and Communications in Medicine (DICOM) image of the current and prior study, study information (e.g., modality, protocol, etc.), contrast usage, modality setting, patient clinical context (e.g., relevant history and physical examination), etc. There are also cases when radiologists would utilize access to the DICOM or console information, which can be shared through accessing the DICOM/console system in real-time, sharing screenshot captured, screen sharing for remote review.

As will be described in greater detail below, in some implementations discussed herein, a radiologist consultation support system is described to provide radiologists with the essential clinical context in a prioritized and summarization fashion. Such implementations may save time, improves quality, and/or mitigates potential errors. One aspect of the radiologist consultation support system is to infer the data needs based on the specific consultation request to ensure access to all the essential data. Then, the radiologist consultation support system presents the prioritized summary data according to the consultation request to enable access to an accurate and holistic view of the patient context efficiently.

In some implementations discussed herein, systems, apparatuses, and methods provide for matching patient data to individual patients. For example, such operations include determining data requirements based on content of a consultation request for a consultation. Missing information is identified based on the determined data requirements by retrieving available data from a data source and identifying unavailable data based on the retrieved available data and the determined data requirements. Summaries are generated by extracting clinical information relevant to performing the consultation from the retrieved available data. The summaries are prioritized and the identified unavailable data is highlighted in context of the consultation request.

In one aspect, a radiologist consultation support system includes an inference module, a missing data module, a summarization module, and a prioritization module. The inference module is to determine data requirements based on content of a consultation request for a consultation. The missing data module is communicatively coupled to the inference module to identify missing information based on the determined data requirements by retrieving available data from a data source module and identify unavailable data based on the retrieved available data and the determined data requirements. The summarization module is communicatively coupled to the missing data module to generate summaries by extracting clinical information relevant to performing the consultation from the retrieved available data. The prioritization module is communicatively coupled to the summarization module to prioritize the summaries and highlight the identified unavailable data in context of the consultation request.

In another aspect, a method for radiologist consultation support includes determining data requirements based on content of a consultation request for a consultation, via an inference module. Missing information is identified based on the determined data requirements by retrieving available data from a data source module and identifying unavailable data based on the retrieved available data and the determined data requirements, via a missing data module communicatively coupled to the inference module. Summaries are generated by extracting clinical information relevant to performing the consultation from the retrieved available data, via a summarization module communicatively coupled to the missing data module. The summaries are prioritized and the identified unavailable data is highlighted in context of the consultation request, via a prioritization module communicatively coupled to the summarization module.

In yet another aspect, at least one machine-readable storage, including a set of instructions, which when executed by a computing device, cause the computing device to determine data requirements based on content of a consultation request for a consultation. Missing information is identified based on the determined data requirements by retrieving available data from a data source and identify unavailable data based on the retrieved available data and the determined data requirements. Summaries are generated by extracting clinical information relevant to performing the consultation from the retrieved available data. The summaries are prioritized and the identified unavailable data is highlighted in context of the consultation request.

In still another aspect, an apparatus for radiologist consultation support includes means for determining data requirements based on content of a consultation request for a consultation. A means for identifying missing information is based on the determined data requirements by retrieving available data from a data source and identifying unavailable data based on the retrieved available data and the determined data requirements. A means for generating summaries is based on extracting clinical information relevant to performing the consultation from the retrieved available data. A means for prioritizing the summaries and highlighting the identified unavailable data operates in context of the consultation request.

It should be appreciated that all combinations of the foregoing concepts and additional concepts discussed in greater detail below (provided such concepts are not mutually inconsistent) are contemplated as being part of the subject matter disclosed herein. In particular, all combinations of claimed subject matter appearing at the end of this disclosure are contemplated as being part of the subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

The various advantages of the embodiments will become apparent to one skilled in the art by reading the following specification and appended claims, and by referencing the following drawings, in which:

FIG. 1 is an illustration of a block diagram of an example radiologist consultation support system according to an embodiment;

FIG. 2 is an illustration of a block diagram of another example radiologist consultation support system according to an embodiment;

FIG. 3 is an illustration of a flowchart of data extraction according to an embodiment;

FIG. 4 is an illustration of a user interface according to an embodiment;

FIG. 5 is an illustration of another user interface according to an embodiment;

FIG. 6 is an illustration of a flowchart of a method for managing radiologist consultations to individual patients according to an embodiment;

FIG. 7 is an illustration of a flowchart of a further method for managing radiologist consultations according to an embodiment;

FIG. 8 is an illustration of a block diagram of a computer program product according to an embodiment;

FIG. 9 is a further illustration of a EMR management system according to an embodiment; and

FIG. 10 is an illustration of a hardware apparatus including a semiconductor package according to an embodiment.

### DETAILED DESCRIPTION

As will be described in greater detail below, in some implementations discussed herein, systems, apparatuses, and methods provide for matching patient data to individual patients. For example, such operations include determining data requirements based on content of a consultation request for a consultation. Missing information is identified based on the determined data requirements by retrieving available data from a data source and identifying unavailable data based on the retrieved available data and the determined data requirements. Summaries are generated by extracting clinical information relevant to performing the consultation from the retrieved available data. The summaries are prioritized and the identified unavailable data is highlighted in context of the consultation request.

Advantageously, some of the implementations of the radiologist consultation support system described herein help to improve the quality and efficiency of communication systems for radiologists. Additionally, or alternatively, the radiologist consultation support system can also be used as a pre-check for order or consultation requests to make sure the relevant and essential data are provided for radiologist to respond to the request.

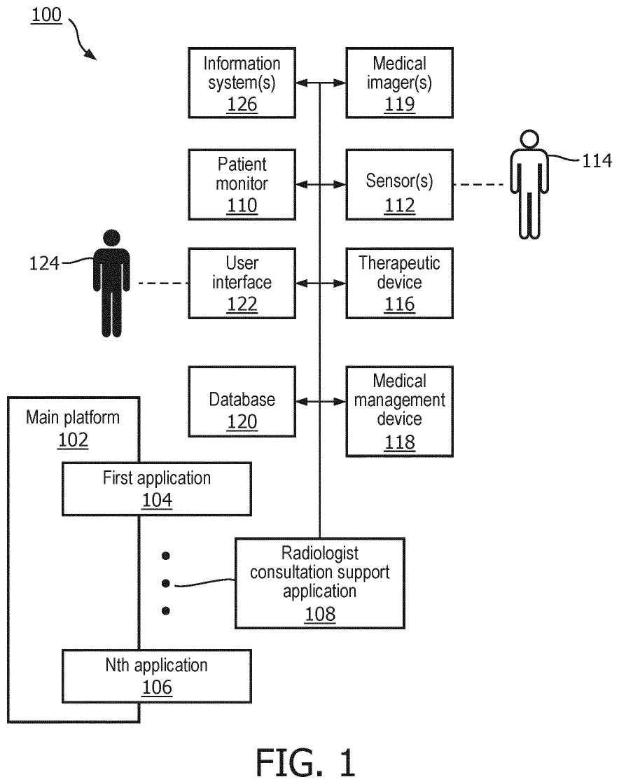

FIG. 1 is an illustration of a block diagram of an example radiologist consultation support system 100 according to an embodiment. For example, the radiologist consultation support system 100 may be centralized or may be distributed and may include some or all elements and components of one or more computers or computer systems.

In the illustrated implementation, the radiologist consultation support system 100 may include a main platform 102 (e.g., also referred to as a care platform herein). In some implementations, the main platform 102 may be embodied as a server computer or a plurality of server computers (e.g., interconnected to form a server cluster, cloud computing resource, the like, and/or combinations thereof).

In some implementations, the main platform 102 is a care platform designed for one or more aspects of patient care. In such an example, the main platform 102 includes one or more of the following care platforms Performance Management Data Platform, Medical Asset Track & Trace System, Virtualized Imaging Solution, Centralized Care Management System, Interoperability Solution, Electronic Medical Record (EMR), radiology information systems (RIS), picture archiving and communication system (PACS), etc. In a patient care setting, there may be many care platforms connected to various clinical and operational data sources (e.g., Health Level Seven (HL7), Fast Healthcare Interoperability Resources (FHIR), machine logs, Real-Time Location System (RTLS), sensors, etc.). As described above, all of these data sources typically only have partial data about individual patients due to the limitations of the technology or platform.

In some implementations, a first application 104 though an Nth application 106, which may include a radiologist consultation support application 108, may be associated with the main platform 102. As will be described in greater detail below, the operations of the radiologist consultation support application 108 provide for matching patient data to individual patients. For example, such operations include determining data requirements based on content of a consultation request for a consultation. Missing information is identified based on the determined data requirements by retrieving available data from a data source and identifying unavailable data based on the retrieved available data and the determined data requirements. Summaries are generated by extracting clinical information relevant to performing the consultation from the retrieved available data. The summaries are prioritized and the identified unavailable data is highlighted in context of the consultation request.

Additionally, or alternatively, the radiologist consultation support system 100 may include a patient monitor 110, a sensor 112 (e.g., one or more sensors 112 that may be associated with a care facility, sensors 112 that may be associated with a patient 114, the like, and/or combinations thereof), a therapeutic device 116, a medical management device 118, medical imager 119, a database 120, a user interface 122 (e.g., one or more user interfaces 122 may be associated with a user 124), an information system 126, the like, and/or combinations thereof. For example, the main platform 102, the patient monitor 110, the sensor 112, the therapeutic device 116, the medical management device 118, medical imager 119, the database 120, the user interface 122, and/or the information system 126, may be in communication with one another via Internet based communicating, cloud based communication, wired communication, wireless communication, the like, and/or combinations thereof.

In an example, the patient monitor 110 may be utilized to access patient data from and/or enter patient data to the database 120. For example, the patient monitor 110 may determine measured patient data (e.g., via one of more of the sensors 112). In such an example, the patient monitor 110 may be configured to monitor a patient for vital signs and the like, and the patient monitor 110 may communicate such measured patient data to the database 120. For example, the sensors 112 may determine dynamic patient condition data including patient vitals (e.g., blood pressure, pulse, temperature, respiration, and/or the like) and/or patient test results (e.g., creatine level, urine flow, potassium level, oxygen saturation, blood glucose level, carbon dioxide level, and/or the like).

In some implementations, the patient monitor 110 may include a bedside-type monitor, a transport-type monitor, a central station-type monitor, the like, and/or combinations thereof.

In some implementations, the sensors 112 may be minimally invasive-style sensors (e.g., by puncturing the skin, sensing through the skin, the like, and/or combinations thereof). Sensors 112 may be wired or wireless.

In another example, the therapeutic device 116 may be utilized to access patient data from and/or enter patient data to the database 120. For example, the therapeutic device 116 may determine measured patient data. In such an example, the therapeutic device 116 may be configured to monitor the delivery of a particular therapy (e.g., a non-medication treatment) to a patient and may communicate such measured patient data to database 120.

In some implementations, the therapeutic device 116 may supply and/or monitor the administration of one or more patient procedures (e.g., dialysis).

In a further example, the medical management device 118 may be utilized to access patient data from and/or enter patient data to the database 120. For example, the medical management device 118 may determine measured patient data. In such an example, the medical management device 118 may be configured to monitor medication delivery to a patient and may communicate such measured patient data to the database 120. In some implementations, the medical management device 118 may supply and/or monitor the administration of one or more patient medications (e.g., blood pressure medications, diuretic medications, anti-anemia medications, cholesterol lowering medications, vitamin supplements, and/or the like).

In some examples, the medical imager 119 include one or more medical imaging devices, medical imaging systems, the like, and/or combinations thereof. For example, the medical imager 119 include a magnetic resonance imaging (MRI) device, an ultrasound device, an x-ray device, a computerized tomography (CT) device, a radiology information systems (RIS), a picture archiving and communication system (PACS), the like, and/or combinations thereof. In such an implementation, the medical imager 119 is associated with standardized format medical imaging information to transmit, store, retrieve, print, process, and display medical imaging information (e.g., Digital Imaging and Communications in Medicine (DICOM) data).

Additionally, or alternatively, in a still further example, the user interface 122 may be utilized to access patient data from and/or enter patient data to the database 120. In some implementations, user interface 122 may be implemented via one or more formfactor devices (e.g., a smart phone, a tablet, a laptop, a workstation, and/or the like), an interface associated with the main platform, and/or an interface associated with the patient monitor 110. Additionally, or alternatively, a care provider (e.g., user 124) may access patient data and/or enter patient data through an analog device, a non-networked patient monitor, a non-networked therapeutic device, a non-networked medical management device, the like, and/or combinations thereof.

In the illustrated implementation, the database 120 may include one or more types of patient data. For example, the database 120 may include patient data including medical images, laboratory result data, microbiology data, medication data, vital sign data, care order data, admission discharge and transfer data, and/or the like. As used herein, the term “database” refers to a collection of data and information organized in such a way as to allow the data and information to be stored, retrieved, updated, and/or manipulated. The term “database” as used herein may also refer to databases that may reside locally or that may be accessed from a remote location (e.g., via remote network servers).

As used herein, the term “patient data” refers to clinical data, imaging, or information related to an individual patient. Patient data may include measured patient data from a medical imaging device, an analog medical device, a sensor, a patient monitor, a therapeutic device, a medical management device, the like, and/or combinations thereof.

In the illustrated implementation, the information system 126 may have or have access to one or more types of patient data that are the same or in addition to the patient data of the database 120. For example, the information system 126 may be a Hospital Information System (HIS). Such a Hospital Information System (HIS) has patient data including Health Level Seven (HL7) data, Fast Healthcare Interoperability Resources (FHIR) data, the like, and/or combinations thereof. Additionally, or alternatively, in some implementations, the information system 126 has or has access to one or more of the following information sources: an Electronic Medical Record (EMR), a radiology information systems (RIS), a picture archiving and communication system (PACS), a Digital Imaging and Communications in Medicine (DICOM), medical imaging devices, a Real-Time Location System (RTLS), sensors, machine logs, a Performance Management Data Platform, a Medical Asset Track & Trace System, a Virtualized Imaging Solution, a Centralized Care Management System, an Interoperability Solution, etc. As described above, all of these data sources typically only have partial data about individual patients due to the limitations of the technology or platform and none have a complete timeline of care on a patient-by-patient basis.

Additionally, or alternatively, in some implementations, the database 120 and/or the information system 126 may include or be associated with a simulated database. In such an example, such a simulated database may generate estimated patient data. For example, the simulated database may utilize some measured patient data from the patient monitor 110, the sensor 112, the therapeutic device 116, the medical management device 118, the medical imager 119, and/or the user interface 122 to generate some other estimated patient data. Such a simulated database may utilize digital twin technology to perform the estimation, for example. In such an example, such estimated patient data may be marked to indicate its estimated nature (rather than measured patient data). Additionally, or alternatively, a weight factor may be applied to the estimated patient data so that the estimated patient data may have a lower weight than corresponding measured patient data.

In some implementations, the radiologist consultation support system 100 may be utilized as an element of an Integrated Clinical Environment (ICE). As used herein, the “Integrated Clinical Environment (ICE)” refers to a platform to create a medical Internet of Things (IOT) associated with the care of a patient. In such an implementation, the radiologist consultation support system 100 may support many real-time clinical decision support algorithms. Additionally, or alternatively, the radiologist consultation support system 100 may support closed loop control algorithms of medical devices in the ICE.

Advantageously, in some implementations, the radiologist consultation support application 108 may help to improve the quality and efficiency of communication systems for radiologists. Additionally, or alternatively, the radiologist consultation support application 108 can also be used as a pre-check for order or consultation requests to make sure the relevant and essential data are provided for radiologist to respond to the request.

As will be described in greater detail below, the radiologist consultation support system 100 and/or the radiologist consultation support application 108 may include several operable components.

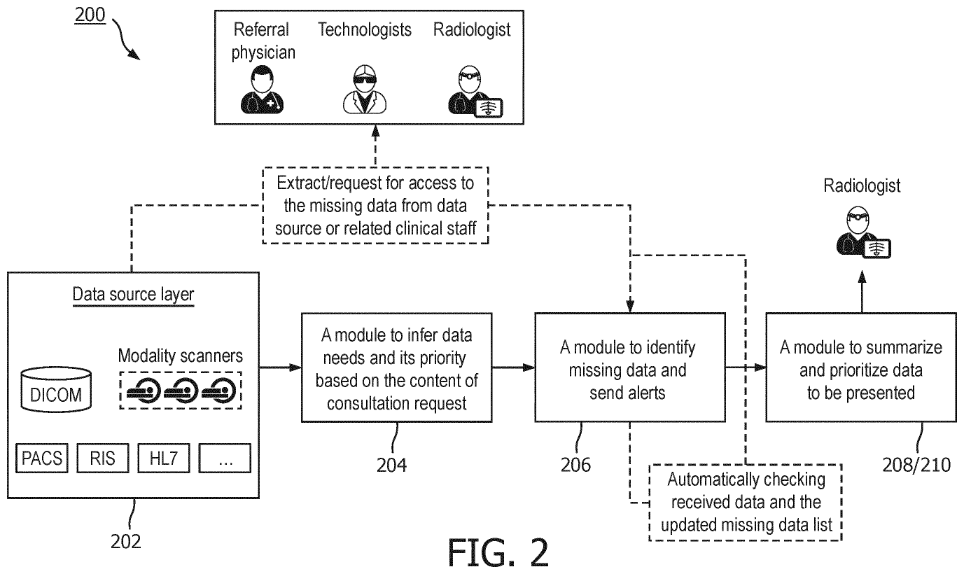

FIG. 2 is an illustration of a block diagram of another example radiologist consultation support system 200 according to an embodiment. As illustrated, the radiologist consultation support system 200 includes a data source module 202, an inference module 204, a missing data module 206, a summarization module 208, and/or a prioritization module 210.

In the illustrated implementation, the data source module 202 is to provide access to an Electronic Medical Record (EMR), a radiology information system (RIS), a picture archiving and communication system (PACS), a medical imaging device, the like, and/or combinations thereof. For example, the data source module 202 may provide access to the medical imager 119, the database 120, the information system 126, the like, and/or combinations thereof (e.g., as discussed above with respect to FIG. 1).

In some examples, the data source module 202 provides access to the relevant data that is typically utilized for radiologists. For example, for patient clinical context, data sources may include patient demographics information (e.g., such as patient weight and height), lab results for biometrics data (e.g., eGFR, blood pressure, etc.), imaging data (e.g., modality scans of the current and prior studies), patient history (e.g., including prior reports/findings), diagnosis findings (e.g., critical and incidental findings), reason for study and urgency, order, the like and/or combinations thereof.

For study information context, data sources may include relevant clinical staff of the study (e.g., technicians, referral physician, etc.), modality, setting of the modality scans, contrast usage, protocol usage, status of patient to indicate if the patient is on the scanner table, screen shot of the console, DICOM images of the prior or the current study, the like and/or combinations thereof.

For consultation log data context, data sources may include if the consultation request is communicated and responded in web-based text format, a log file might be generated to store the communication.

In some examples, the inference module 204 is to determine data requirements based on content of a request for a consultation.

In some implementations, the inference module 204 is to infer data needs based on the content of the consultation request. For example, the inference module 204 may automatically infer the data needs of a radiologist to respond to a consultation request. The output of the inference module 204 is a list (or other compilation) of data required tailored based on the content of consultation request, for example.

For example, the data required to provide appropriate consultation may vary depending on the specific consultation request task. Such consultation request tasks can be classified into a few categories, including image check, protocolling, study review, order question, procedure questions, etc. For protocolling, radiologists often utilize patient demographics information (e.g., height, weight), lab data (e.g., eGFR), patient's current and prior clinical context to get a holistic view of the patient. For image quality check, a radiologist would typically utilize access to the DICOM images of the current and prior studies, study information (e.g., modality, protocol, etc.), contrast usage, modality setting, patient clinical context (e.g., relevant history and physical examination), etc.

In some examples, data needs can thus be inferred by the inference module 204 based on the consultation request (e.g., the type of consultation request category and/or task). To automatically infer the data needs, the consultation category could be identified first by the inference module 204, which in most cases may be indicated directly. In some examples, a pre-canned consultation content label could be provided for this purpose. For example, if a technologist asks the radiologist to check the image quality before releasing the patient, a label ‘image quality check’ could be given as direct input for the inference module 204. Additionally, or alternatively, one or more artificial intelligence (AI) models could be trained to categorize the consultation automatically. Using the previous example, a technologist could ask, “could you please review the image?” In such cases, a natural language processing (NLP) based classification model could be used to identify the content automatically. In such an example, the inference module 204 may first tokenize the sentence and remove less informative words such as ‘please,’ ‘you,’ and ‘the.’ Then, the word data may be transformed by the inference module 204 into a vector presentation used as the input to the NLP model. The inference module 204 might then assign a consultation category for the request in response to the output of the NLP model.

In some implementations, the missing data module 206 is communicatively coupled to the data source module 202 and the inference module 204. For example, the missing data module 206 is to identify missing information based on the determined data requirements by retrieving available data from the data source module 202 and identify unavailable data based on the retrieved available data and the determined data requirements.

In some examples, the missing data module 206 is to identify missing data. For example, missing information is identified based on the data needs to perform the specific consultation task and requests for data access are sent. Missing data would be outlined when presenting to a user to inform the radiologists that only partial data is provided, for example.

In some examples, the missing data module 206 is to send an alert of data availability to the radiologist. For example, with such a data availability alert, a radiologists can decide if they are able to respond to the consultation request using existing data.

In some implementations, the summarization module 208 is communicatively coupled to the missing data module 206. For example, the summarization module 208 is to generate summaries by extracting clinical information relevant to performing the consultation from the retrieved available data.

In some examples, the prioritization module 210 is communicatively coupled to the summarization module 208. For example, the prioritization module 210 is to prioritize the summaries and highlight the identified unavailable data in context of the consultation request.

For example, the summarization module 208 is to summarize and the prioritization module 210 is to prioritize data to be presented. For example, summarization of key clinical information is extracted from the data and the presentation is prioritized based on the clinical relevance to perform the consultation.

In operation, the radiologist consultation support system 200 may help to improve the quality and efficiency in clinical communication and collaboration. In some implementations, radiologist consultation support system 200 can be offered as a stand-alone application or can be integrated with clinical and collaboration applications (e.g., a Remote Command Center, or the like) and/or with other hospital information systems. Some examples herein can be implemented on a user interface (UI) level of hospital informatics systems (e.g., PACS or the like), clinical and collaboration systems (e.g., a Radiology Operations Command Center (ROCC) platform or the like), the like, and/or combinations thereof.

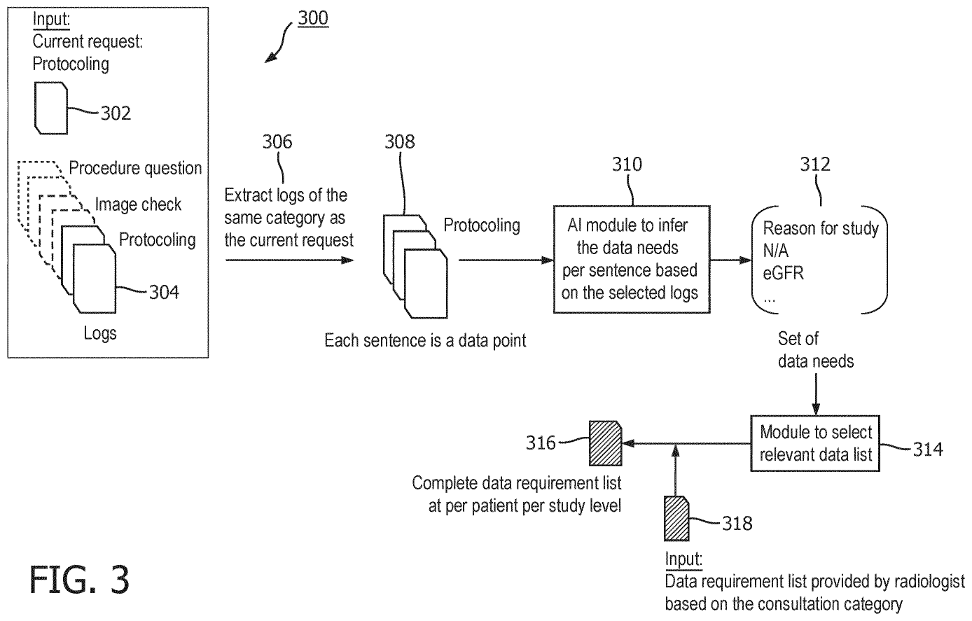

FIG. 3 is an illustration of a data extraction flowchart 300 according to an embodiment. As illustrated, data extraction flowchart 300 shows an example of extraction of a data requirement list based on the content of consultation request (e.g., as might be output by the inference module 204 of FIG. 2).

In some implementations, another AI model (e.g., in addition to or instead of the AI model discussed in FIG. 2 with respect to the inference module 204) could be trained to learn the data request at per patient and per study level to provide a more complete, accurate, and patient-specific overview. For example, as there is a limited set of consultation request categories, radiologists could provide data access requirements per category. Accordingly, data needs could be inferred from multiple data sources, which may include but is not limited to communication logs, any log of DICOM image review, logs of information access requests, the like, and/or combinations thereof.

For example, the communication log may keep track of all the communication history. If information is missing, there would very likely be conversations around missing data and request for access, which could be logged. Logs of DICOM image review may be utilized to infer the need for image data, as they indicate if DICOM image would be needed or to indicate which DICOM image would be needed for a specific case. The log which tracks information access in other hospital system (e.g., electronic health records (HER) system, electronic medical records (EMR) system) could also be utilized for such data needs. Most data needs information could be conveyed through a communication history. The inference module 204 (illustrated in FIG. 2) can consume any of the data sources, single or in combination as the data input and the output and performance of to the inference module 204 (illustrated in FIG. 2) would vary accordingly.

In some examples, at processing block 306, with the consultation category of the current protocolling request identified, the radiologist consultation support system 200 (illustrated in FIG. 2) may extract all the logs 308 from the same consultation category of the current request 302 found in the communication log of all the communication history 304, which would be used as the input of the AI module.

At processing block 310, the AI module may process each data point, learned the context of the study and output the required data fields.

At processing block 312, the output of the AI module would be a list of data indicated as required by the log document.

At processing block 314, a subset of data could be selected as certain data needs could be very specific to the training data and not applied in the current context.

At processing block 316, combining the selected data list with the input data requirement 318 provided by the radiologists, the output data requirement is generated and tailored based on the content of the current consultation request. In some examples, the data requirement list is not restricted to text-based data and may cover image data and/or the like. For example, for image quality check consultations, relevant prior images might be utilized.

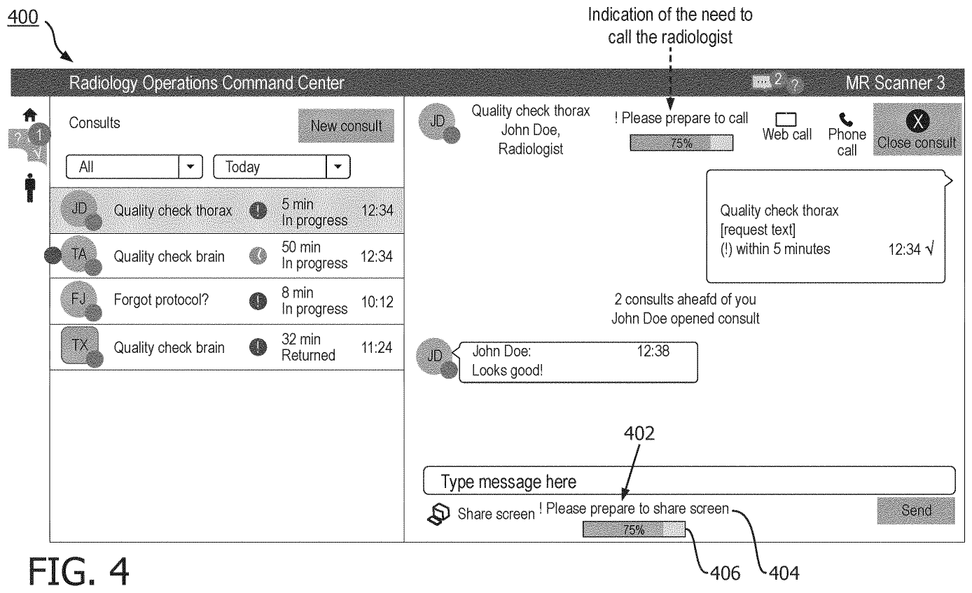

FIG. 4 is an illustration of a user interface 400 according to an embodiment. As illustrated, the radiologist consultation support system 200 (illustrated in FIG. 2) may also include a screensharing module (not illustrated) to infer the need for screensharing. Such a screensharing module could provide an indication, such as illustrated in example user interface 400, to the question initiator to communicate the potential need to call or start a screensharing, for example. For example, there may be cases when radiologists would prefer to access study/patient information or communicate with the question initiators (e.g., technologists, referral physicians, etc.) through call or screensharing, in which cases the question initiators should expect to connect with the radiologists, or to initiate a call/screensharing.

As illustrated, the user interface 400 includes an indication 402 shown to the question initiator (e.g., a technologists in this case) of the potential need to start a call/screen sharing with the radiologists for a particular consultation request. The indication 402 may include a text message 404 to show the action to be expected and the number indicator 406 to show the likelihood of requiring call/screen sharing from the radiologists. Advantageously, such an indication 402 may enable the question initiator to be prepared or act proactively.

In some implementations, the indicator 402 can be realized by a naive frequency based algorithm. For example, with the question category identified, the percentage of consultation requests is calculated in which a call or screensharing was established. The percentage would be used to indicate the likelihood for initiate a call or a screensharing in this case, which correspond to the number shown in under the indicator message.

In some examples, a threshold can be set up to determine the message to be shown in the indicator. For example, if more than 70% of consultation requests in this question category would require a call/screen sharing, then messages are shown (e.g., such as ‘please prepare to call/share screen’; otherwise, no indication would be presented).

In some implementations, the indication 402 could also be trained using AI modules. For example, the training label would be a binary output representing if a call/screensharing was initiated in this case. In such an example, dependent data would be treated as attributes of the consultation requests, which might include but are not limited to patient status, question category, patient context, the like, and/or combinations thereof. The output of which would be the likelihood of initiating a call/screensharing, in this example. For example, a logistic regression would be suitable for such implementations.

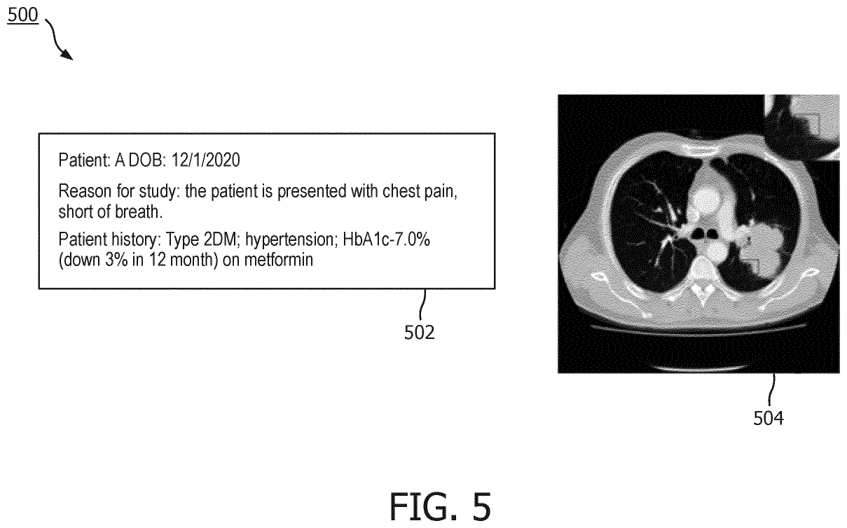

FIG. 5 is an illustration of another example summarization user interface 500 according to an embodiment. As illustrated, the summarization module 208 and/or the prioritization module 210 of the radiologist consultation support system 200 (illustrated in FIG. 2) may output the summarization user interface 500 to provide a summarization of the clinical context.

In some example, the summarization user interface 500 includes a text summary 502 of the clinical context. For example, based on the data type, clinical data could be classified as non-text, text-based, and image data. Text-based data could be further classified as free text and codified text data, which uses pre-defined coding to represent a text field. For example, code ‘LV001’ may be used to represent a left ventricular hypertrophy. Most of the clinical contexts may be provided in the format of free-text or codified text, such as diagnostic findings, patient history, the reason for study, etc. via the text summary 502.

In some example, the summarization user interface 500 includes an image summary 504. Modality scans are imaging data, which contain rich clinical information but could be excess for radiologists to review. Thus, the image summary 504 of the clinical status would help radiologists to have a quick overview of the clinical context of the case. Various AI models could be trained for implementation.

Additionally, or alternatively, upon clicking on the summary data in the summarization user interface 500, a radiologists would have access to the full data source from which the summary is extracted.

In some implementation, prioritization is focused on presenting the most relevant data first and leverage smart visualization to present the data so that information could be better consumed. For example, such prioritization may be based on the context of the consultation request. In such an example, if the consultation request is for ‘image check,’ summarization should be focused on image quality and prioritize key images with artifacts for radiologists to review. On the other hand, if the request is for ‘protocolling’, then the summarization module 208 (illustrated in FIG. 2) may provide a holistic summary of the patient's clinical context and the prioritization module 210 (illustrated in FIG. 2) may prioritize key reasons for study so that radiologists could understand the diagnostic focus of the case. If the request is for ‘initial review before releasing,’ the summarization module 208 and/or the prioritization module 210 (illustrated in FIG. 2) may summarize and prioritize the critical findings on the imaging data.

Additionally, or alternatively, the summarization module 208 and/or the prioritization module 210 (illustrated in FIG. 2) are to present suggested potential follow-up actions to be taken based on the summary data. For example, instead of typing a response manually, radiologists can select from one or more automatically generated reply messages for quick reply. For example, if important information are indicated as missing, ‘please provide more information’ could be presented in the summarization user interface 500 or other user interface. Other quick reply message options could be ‘release patient’, ‘do extra contrast scan’, ‘schedule PET CT,’ the like, and/or combinations thereof. In some examples, the most frequently used actions could be provided as quick reply message options and/or one or more AI modules could be trained to provide such quick reply message options.

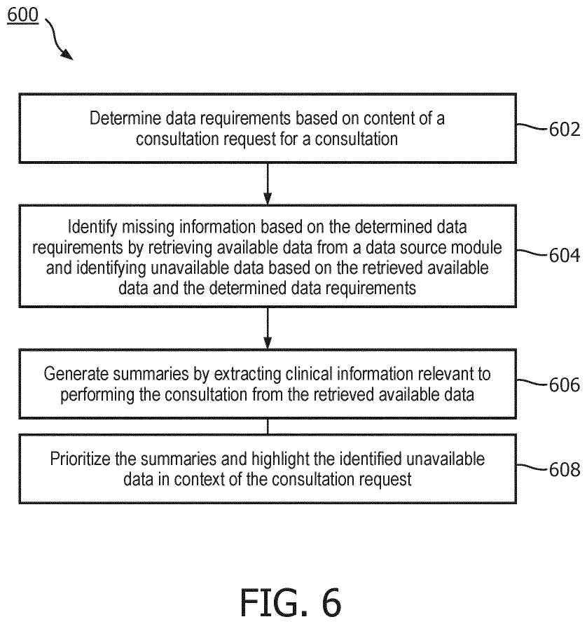

FIG. 6 shows an example method 600 for managing radiologist consultations according to an embodiment. The method 600 may generally be implemented in the radiologist consultation support system 100 (FIG. 1) and/or the radiologist consultation support system 200 (FIG. 2), already discussed.

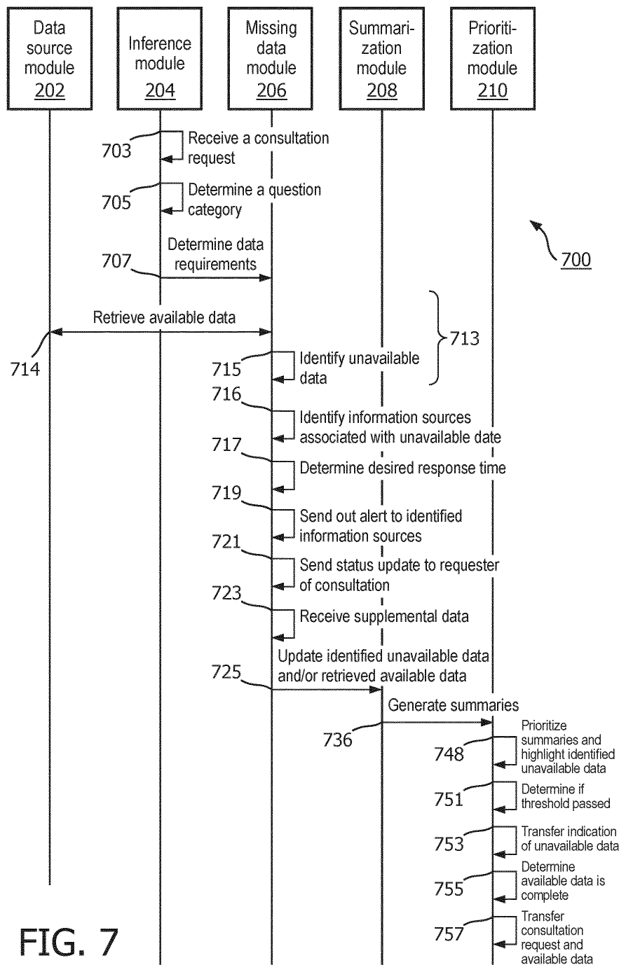

In an embodiment, the method 600 (as well as method 700 (FIG. 7) may be implemented in logic instructions (e.g., software), configurable logic (e.g., firmware), fixed-functionality hardware logic (e.g., hardware), etc., or any combination thereof.

Illustrated processing block 602 provides for determining data requirements. For example, an inference module may determine data requirements based on content of a consultation request for a consultation.

Illustrated processing block 604 provides for identifying missing information For example, a missing data module communicatively coupled to the inference module may identify missing information based on the determined data requirements by retrieving available data from a data source module and identify unavailable data based on the retrieved available data and the determined data requirements.

Illustrated processing block 606 provides for generating summaries. For example, a summarization module communicatively coupled to the missing data module may generate summaries by extracting clinical information relevant to performing the consultation from the retrieved available data.

Illustrated processing block 608 provides for prioritizing the summaries and highlighting the identified unavailable data. For example, a prioritization module communicatively coupled to the summarization module may prioritize the summaries and highlight the identified unavailable data in context of the consultation request.

In some examples, the methods described herein (e.g., method 600 and/or method 700) may be performed at least in part by cloud processing.

It will be appreciated that some or all of the operations described herein (e.g., method 600 and/or method 700) that have been described using a “pull” architecture (e.g., polling for new information followed by a corresponding response) may instead be implemented using a “push” architecture (e.g., sending such information when there is new information to report), and vice versa.

Additional and/or alternative operations for method 600 are described in greater detail below in the description of FIG. 7.

FIG. 7 is a flowchart of an example of another method 700 for managing radiologist consultations according to an embodiment. The method 700 may generally be implemented in the radiologist consultation support system 100 (FIG. 1) and/or the radiologist consultation support system 200 (FIG. 2), already discussed.

As illustrated, various processing blocks are illustrated as being performed by the data source module 202, the inference module 204, the missing data module 206, the summarization module 208, and/or the prioritization module 210 in conjunction with one another (e.g., as discussed above in FIG. 2).

Illustrated processing block 703 provides for reception of a consultation request. For example, an inference module may receive a consultation request.

Illustrated processing block 705 provides for determine a question category. For example, the inference module may determine a question category in response to reception of the consultation request.

In some implementations, the determination of the question category is performed based at least in part on a type of workflow associated with the consultation request. For example, the type of workflow is an image quality check, protocolling to confirm pertinence of a request, a study review, a procedure question, answering an order question, a protocol procedure request, the like, and/or combinations thereof.

As used herein, the term “image quality check” refers to a request to a radiologist to eyeball a radiological image and assess if it has diagnostic quality. If not, the image needs to be retaken.

As used herein, the term “protocolling” refers to assigning an imaging protocol to an imaging order of a referring physician. Sometimes, referral providers don't quite know what to order and their orders might be misguided given the symptoms/history of the patient. In that case a radiologist may make a judgment call.

As used herein, the term “study review” refers to a radiologist review of an image right after acquisition to ensure that there are no findings that require add-on imaging. In that case, the patient may stay on the table or in the hospital for follow-up imaging.

As used herein, the term “answering an order question” refers to respond to a referring provider who is entering an image order on what examination to select.

Illustrated processing block 707 provides for determining data requirements. For example, the inference module may determine data requirements based on content of a consultation request for a consultation.

In some examples, the determination of the data requirements is based at least in part on the question category, for example.

In some implementations, the determination of the data requirements is further based at least in part on information from one or more previous consultation requests.

Illustrated processing block 713 provides for identifying missing information. For example, a missing data module communicatively coupled to the inference module may identify missing information based on the determined data requirements by retrieving available data at processing block 714 from the data source module and identify unavailable data at processing block 715 based on the retrieved available data and the determined data requirements.

In some implementations, the data source module is to provide access to an Electronic Medical Record (EMR), a radiology information system (RIS), a picture archiving and communication system (PACS), a medical imaging device, the like, and/or combinations thereof.

Illustrated processing block 716 provides for identifying an information source. For example, the missing data module may identify an information source associated with the identified unavailable data Illustrated processing block 717 provides for determining a desired response time frame. For example, the missing data module may determine a desired response time frame based at least in part on an assessment of an urgency of the consultation request.

Illustrated processing block 719 provides for sending out an alert. For example, the missing data module may send out an alert to the identified information source, where the alert includes an indication of the identified unavailable data and the desired response time frame.

Illustrated processing block 721 provides for sending out a status updates. For example, the missing data module may send out a status update to a sender of the consultation request. In such an example, the status update may include an indication of how likely it is that supplemental data is needed in response to the alert indication of the identified unavailable data, an indication of the identified information source from which the supplemental data is being requested, and/or an indication of the desired response time frame.

Illustrated processing block 723 provides for receiving supplemental data. For example, the missing data module may receive supplemental data from the identified information source in response to the alert indication of the identified unavailable data.

Illustrated processing block 725 provides for updating the identified unavailable data. For example, the missing data module may update the identified unavailable data (e.g., as well as the retrieved available data).

Illustrated processing block 736 provides for generating summaries. For example, a summarization module communicatively coupled to the missing data module may generate summaries by extracting clinical information relevant to performing the consultation from the retrieved available data.

Illustrated processing block 748 provides for prioritizing the summaries and highlighting the identified unavailable data. For example, a prioritization module communicatively coupled to the summarization module may prioritize the summaries and highlight the identified unavailable data in context of the consultation request.

Illustrated processing block 751 provides for determine that any remaining identified unavailable data passes a threshold. For example, the prioritization module may determine that any remaining identified unavailable data passes a threshold.

Illustrated processing block 753 provides for transfer an indication of the remaining identified unavailable data. For example, the prioritization module may transfer the consultation request, available data, and/or an indication of the remaining identified unavailable data to a receiver associated with a radiologist in response to the determination that the threshold has been passed.

Illustrated processing block 755 provides for determining that the available data is complete. For example, the prioritization module may determine that there is no remaining identified unavailable data and that available data is complete.

Illustrated processing block 757 provides for transferring the consultation request and the available data. For example, the prioritization module may transfer the consultation request and the available data to a receiver associated with a radiologist in response to the determination that the available data is complete.

In some implementations, the prioritization module may transfer the consultation request and the available data to a user interface associated with an assigned radiologist.

Additionally, or alternatively, the procedures described herein may provide a framework for clinical deployment of decision support algorithms. These procedures can work together with many clinical decision support (CDS) algorithms (such as acute kidney injury (AKI), acute respiratory distress syndrome (ARDS), acute decompensated heart failure (ADHF), etc.). Clinical decision support (CDS) refers to computer-based support of clinical staff responsible for making decisions for the care of patients. Computer-based support for clinical decision-making staff may take many forms, from patient-specific visual/numeric health status indicators to patient-specific health status predictions and patient-specific health care recommendations. Further, the procedures described herein may be deployed on analytics platforms (such as Inference Engine, Critical Care Information System, Interoperability Solution, etc.) in conjunction with CDS algorithms.

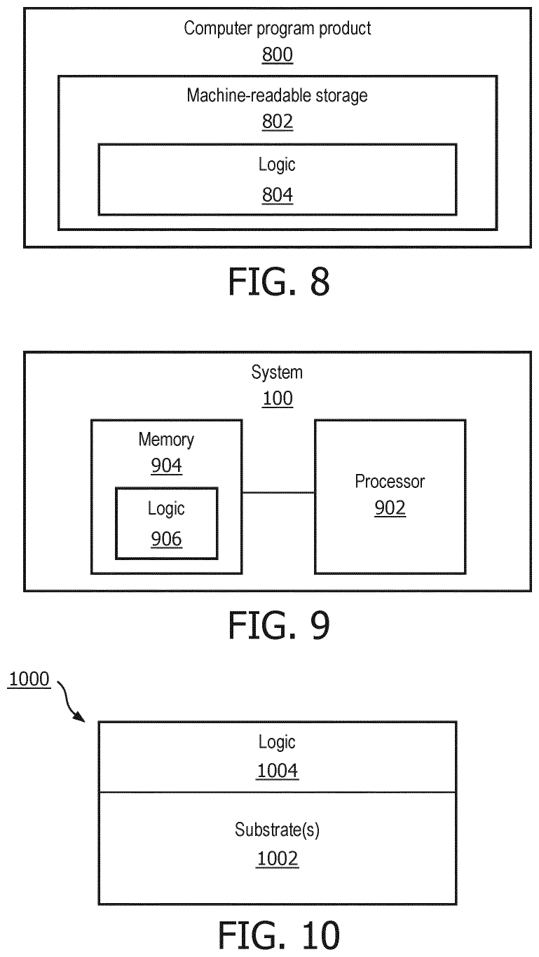

FIG. 8 illustrates a block diagram of an example computer program product 800. In some examples, as shown in FIG. 8, computer program product 800 includes a machine-readable storage 802 that may also include logic 804. In some implementations, the machine-readable storage 802 may be implemented as a non-transitory machine-readable storage. In some implementations the logic 804 may be implemented as machine-readable instructions, such as software, for example. In an embodiment, the logic 804, when executed, implements one or more aspects of the method 600 (FIG. 6), the method 700 (FIG. 7), and/or realize the system 100 (FIG. 1 and/or FIG. 2), already discussed.

FIG. 9 shows an illustrative example of the radiologist consultation support system 100. In the illustrated example, the radiologist consultation support system 100 may include a processor 902 and a memory 904 communicatively coupled to the processor 902. The memory 904 may include logic 906 as a set of instructions. In some implementations the logic 906 may be implemented as software. In an embodiment, the logic 906, when executed by the processor 902, implements one or more aspects of the method 600 (FIG. 6), the method 700 (FIG. 7), and/or realize the system 100 (FIG. 1 and/or FIG. 2), already discussed.

In some implementations, the processor 902 may include a general purpose controller, a special purpose controller, a storage controller, a storage manager, a memory controller, a micro-controller, a general purpose processor, a special purpose processor, a central processor unit (CPU), the like, and/or combinations thereof.

Further, implementations may include distributed processing, component/object distributed processing, parallel processing, the like, and/or combinations thereof. For example, virtual computer system processing may implement one or more of the methods or functionalities as described herein, and the processor 902 described herein may be used to support such virtual processing.

In some examples, the memory 904 is an example of a computer-readable storage medium. For example, memory 904 may be any memory which is accessible to the processor 902, including, but not limited to RAM memory, registers, and register files, the like, and/or combinations thereof. References to “computer memory” or “memory” should be interpreted as possibly being multiple memories. The memory may for instance be multiple memories within the same computer system. The memory may also be multiple memories distributed amongst multiple computer systems or computing devices.

FIG. 10 shows an illustrative semiconductor apparatus 1000 (e.g., chip and/or package). The illustrated semiconductor apparatus 1000 includes one or more substrates 1002 (e.g., silicon, sapphire, or gallium arsenide) and logic 1004 (e.g., configurable logic and/or fixed-functionality hardware logic) coupled to the substrate(s) 1002. In an embodiment, the logic 1004 implements one or more aspects of the method 600 (FIG. 6), the method 700 (FIG. 7), and/or realize the system 100 (FIG. 1 and/or FIG. 2), already discussed.

In some implementations, logic 1004 may include transistor array and/or other integrated circuit/IC components. For example, configurable logic and/or fixed-functionality hardware logic implementations of the logic 1004 may include configurable logic such as, for example, programmable logic arrays (PLAs), field programmable gate arrays (FPGAs), complex programmable logic devices (CPLDs), or fixed-functionality logic hardware using circuit technology such as, for example, application specific integrated circuit (ASIC), complementary metal oxide semiconductor (CMOS) or transistor-transistor logic (TTL) technology, the like, and/or combinations thereof.

All definitions, as defined and used herein, should be understood to control over dictionary definitions, definitions in documents incorporated by reference, and/or ordinary meanings of the defined terms.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. The term “coupled” may be used herein to refer to any type of relationship, direct or indirect, between the components in question, and may apply to electrical, mechanical, fluid, optical, electromagnetic, electromechanical, or other connections. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components.

In the claims, as well as in the specification above, the terms “first”, “second”, etc. may be used herein only to facilitate discussion, and carry no particular temporal or chronological significance unless otherwise indicated.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The indefinite articles “a” and “an,” as used herein in the specification and in the claims, unless clearly indicated to the contrary, should be understood to mean “at least one.” As used herein, the term “or” or “and/or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

As used in this application and in the claims, a list of items joined by the term “one or more of” may mean any combination of the listed terms. For example, the phrases “one or more of A, B or C” may mean A; B; C; A and B; A and C; B and C; or A, B and C.

As is described above in greater detail, one or more processor, other unit, the like, and/or combinations thereof may fulfill the functions of several items recited in the claims.

As is described above in greater detail, a computer program may be stored/distributed on a suitable computer readable medium, such as an optical storage medium or a solid-state medium supplied together with or as part of other hardware, but may also be distributed in other forms, such as via the Internet or other wired or wireless telecommunication systems.

It should also be understood that, unless clearly indicated to the contrary, in any methods discussed herein that include more than one step or act, the order of the steps or acts of the method is not necessarily limited to the order in which the steps or acts of the method are recited. Further, such methods may include additional or alternative steps or acts.

As used in the claims, the mere fact that certain measures are recited in mutually different dependent claims does not indicate that a combination of these measured cannot be used to advantage.

It is also noted that the claims may include reference signs/numerals in accordance with PCT Rule 6.2(b). However, the present claims should not be considered to be limited to the exemplary embodiments corresponding to the reference signs/numerals.

Those skilled in the art will appreciate from the foregoing description that the broad techniques of the embodiments of the present invention can be implemented in a variety of forms. Therefore, while the embodiments of this invention have been described in connection with particular examples thereof, the true scope of the embodiments of the invention should not be so limited since other modifications will become apparent to the skilled practitioner upon a study of the drawings, specification, and following claims.

1. A radiologist consultation support system, comprising:
an inference module to determine data requirements based on content of a consultation request for a consultation;
a missing data module communicatively coupled to the inference module, the missing data module to identify missing information based on the determined data requirements by retrieving available data from a data source module and identify unavailable data based on the retrieved available data and the determined data requirements;
a summarization module communicatively coupled to the missing data module, the summarization module to generate summaries by extracting clinical information relevant to performing the consultation from the retrieved available data; and
a prioritization module communicatively coupled to the summarization module, the prioritization module to prioritize the summaries and highlight the identified unavailable data in context of the consultation request.

2. The radiologist consultation support system of claim 1, wherein the inference module is further to determine a question category in response to reception of the consultation request, wherein the determination of the data requirements is based on the question category.

3. The radiologist consultation support system of claim 2, wherein the determination of the data requirements is further based on information from one or more previous consultation requests.

4. The radiologist consultation support system of claim 2, wherein the determination of the question category is performed based on a type of workflow associated with the consultation request, and wherein the type of workflow is one or more of an image quality check, protocolling to confirm pertinence of a request, a study review, a procedure question, answering an order question, and a protocol procedure request.

5. The radiologist consultation support system of claim 1, further comprising the data source module, wherein the data source module is to provide access to one or more of an Electronic Medical Record (EMR), a radiology information system (RIS), a picture archiving and communication system (PACS), and a medical imaging device.

6. The radiologist consultation support system of claim 1, wherein the missing data module is further to:
identify an information source associated with the identified unavailable data;
determine a response time frame based on an assessment of an urgency of the consultation request; and
send out an alert to the identified information source, wherein the alert includes an indication of the identified unavailable data and the response time frame.

7. The radiologist consultation support system of claim 6, wherein the missing data module is further to:
send out a status update to a sender of the consultation request, wherein the status update includes an indication of how likely it is that supplemental data is needed in response to the indication of the identified unavailable data of the alert, an indication of the identified information source from which the supplemental data is being requested, and an indication of the response time frame.

8. The radiologist consultation support system of claim 6, wherein the missing data module is further to:
receive supplemental data from the identified information source in response to the indication of the identified unavailable data of the alert; and
update the identified unavailable data.

9. The radiologist consultation support system of claim 8, wherein the prioritization module further to:
determine that there is no remaining identified unavailable data and that available data is complete; and
transfer the consultation request and the available data to a receiver associated with a radiologist in response to the determination that the available data is complete.

10. The radiologist consultation support system of claim 8, wherein the prioritization module further to:
determine that any remaining identified unavailable data passes a threshold; and
transfer the consultation request, available data, and an indication of the remaining identified unavailable data to a receiver associated with a radiologist in response to the determination that the threshold has been passed.

11. A method for radiologist consultation support, comprising:
determining, via an inference module, data requirements based on content of a consultation request for a consultation;
identifying, via a missing data module communicatively coupled to the inference module, missing information based on the determined data requirements by retrieving available data from a data source module and identifying unavailable data based on the retrieved available data and the determined data requirements;
generating, via a summarization module communicatively coupled to the missing data module, summaries by extracting clinical information relevant to performing the consultation from the retrieved available data; and
prioritizing, via a prioritization module communicatively coupled to the summarization module, the summaries and highlighting the identified unavailable data in context of the consultation request.

12. The method for radiologist consultation support of claim 11, further comprising determining, via the inference module, a question category in response to reception of the consultation request, wherein the determination of the data requirements is based on the question category, wherein the determination of the data requirements is further based on information from one or more previous consultation requests, wherein the determination of the question category is performed based on a type of workflow associated with the consultation request, and wherein the type of workflow is one or more of an image quality check, protocolling to confirm pertinence of a request, a study review, a procedure question, answering an order question, and a protocol procedure request.

13. The method for radiologist consultation support of claim 11, further comprising:
identifying, via the missing data module, an information source associated with the identified unavailable data;
determining, via the missing data module, a response time frame based on an assessment of an urgency of the consultation request;
sending, via the missing data module, out an alert to the identified information source, wherein the alert includes an indication of the identified unavailable data and the response time frame;
sending, via the missing data module, out a status update to a sender of the consultation request, wherein the status update includes an indication of how likely it is that supplemental data is needed in response to the indication of the identified unavailable data of the alert, an indication of the identified information source from which the supplemental data is being requested, and an indication of the response time frame;
receiving, via the missing data module, supplemental data from the identified information source in response to the indication of the identified unavailable data of the alert; and
updating, via the missing data module, the identified unavailable data.

14. The method for radiologist consultation support of claim 13, further comprising:
determining, via the prioritization module, that there is no remaining identified unavailable data and that available data is complete; and
transferring, via the prioritization module, the consultation request and the available data to a receiver associated with a radiologist in response to the determination that the available data is complete.

15. The method for radiologist consultation support of claim 13, further comprising:
determining, via the prioritization module, that any remaining identified unavailable data passes a threshold; and
transferring, via the prioritization module, the consultation request, available data, and an indication of the remaining identified unavailable data to a receiver associated with a radiologist in response to the determination that the threshold has been passed.

16. At least one machine-readable storage, comprising a set of instructions, which when executed by a computing device, cause the computing device to:
determine data requirements based on content of a consultation request for a consultation;
identify missing information based on the determined data requirements by retrieving available data from a data source and identify unavailable data based on the retrieved available data and the determined data requirements;
generate summaries by extracting clinical information relevant to performing the consultation from the retrieved available data; and
prioritize the summaries and highlight the identified unavailable data in context of the consultation request.

17. The at least one machine-readable storage of claim 16, wherein the set of instructions, which when executed by the computing device, cause the computing device further to:
determine a question category in response to reception of the consultation request, wherein the determination of the data requirements is based on the question category, wherein the determination of the data requirements is further based on information from one or more previous consultation requests, wherein the determination of the question category is performed based on a type of workflow associated with the consultation request, and wherein the type of workflow is one or more of an image quality check, protocolling to confirm pertinence of a request, a study review, a procedure question, answering an order question, and a protocol procedure request.

18. The at least one machine-readable storage of claim 16, wherein the set of instructions, which when executed by the computing device, cause the computing device further to:
identify an information source associated with the identified unavailable data;
determine a response time frame based on an assessment of an urgency of the consultation request;
send out an alert to the identified information source, wherein the alert includes an indication of the identified unavailable data and the response time frame;
send out a status update to a sender of the consultation request, wherein the status update includes an indication of how likely it is that supplemental data is needed in response to the indication of the identified unavailable data of the alert, an indication of the identified information source from which the supplemental data is being requested, and an indication of the response time frame;
receive supplemental data from the identified information source in response to the indication of the identified unavailable data of the alert; and
update the identified unavailable data.

19. The at least one machine-readable storage of claim 18, wherein the set of instructions, which when executed by the computing device, cause the computing device further to:
determine that there is no remaining identified unavailable data and that available data is complete; and
transfer the consultation request and the available data to a receiver associated with a radiologist in response to the determination that the available data is complete.

20. The at least one machine-readable storage of claim 18, wherein the set of instructions, which when executed by the computing device, cause the computing device further to:
determine that any remaining identified unavailable data passes a threshold; and
transfer the consultation request, available data, and an indication of the remaining identified unavailable data to a receiver associated with a radiologist in response to the determination that the threshold has been passed.
